(19) United States
(12) Patent Application Publication
(10) Pub. No.: US 2024/0050161 A1
(43) Pub. Date: Feb. 15, 2024

(54) ACTIVE NAVIGATION SYSTEM OF SURGERY AND CONTROL METHOD THEREOF

(71) Applicant: SHENZHEN RESEARCH INSTITUTE OF NANKAI UNIVERSITY, Shenzhen, Guangdong (CN)

(72) Inventors: Yanding Qin, Tianjin (CN); Jianda HAN, Tianjin (CN); Hongpeng WANG, Tianjin (CN); Yugen YOU, Tianjin (CN); Zhichao SONG, Tianjin (CN); Yiyang MENG, Tianjin (CN)

(21) Appl. No.: 18/268,316

(22) PCT Filed: Aug. 1, 2022
(86) PCT No.: PCT/CN2022/109446

§ 371 Date: Jun. 20, 2023

(30) Foreign Application Priority Data
Jul. 7, 2021 (CN) 202110764801.5

(51) Int. Cl. A61B 34/20; A61B 34/30; A61B 34/00

(52) U.S. Cl. CPC A61B 34/20; A61B 34/30; A61B 34/70; A61B 2034/2059

(57) ABSTRACT

An active navigation system of a surgery and a control method include: Step 1, measurement viewing angle multi-objective optimization: inputting position parameters of the positioning tools and setting other related parameters, and solving a set of optimal measurement viewing angles through multi-objective optimization; Step 2, a multi-objective decision of a pose of the robot: according to the set of optimal measurement viewing angles, recommending, to a user, an optimal pose scheme of the robot in each link of the surgery by using a multi-objective decision algorithm; or selecting the optimal pose scheme of the robot in each link of the surgery; and Step 3, planning and execution of a path of the robot: according to the selected optimal pose scheme of the robot in each link of the surgery, planning the path of the robot from the current pose to the optimal pose scheme.

### TECHNICAL FIELD

The present disclosure relates to the technical field of medical equipment, in particular to the field of surgical robots, and in particular to an active navigation system of a surgery and a control method thereof.

### BACKGROUND

With the help of image navigation technology, the robot-assisted surgery system can accurately position the surgical site and operating tools to assist doctors in carrying out minimally invasive surgery, remote surgery or robot-assisted surgery. At present, surgical navigation relies on an optical navigation device to position the surgical site or the surgical tool by observing and identifying the optical positioning tool and calculating the image and the position. In the practical operation, the surgical navigation device is manually adjusted by the doctor who assists the surgery according to the surgical needs. Specifically, by dragging the handle of the device, the optical navigation device is adjusted to the appropriate observation position. However, this interactive method brings a lot of inconvenience in the practical surgical process. For some special surgical position designs, it is difficult to adjust the appropriate measurement position by hand alone, and the position accuracy cannot be guaranteed.

It has become a new trend to give motion capability to the optical navigation device. It is necessary to realize the active navigation of optical navigation, which requires the robot not only to have optical navigation sensors for positioning, but also to have sensors with other environmental sensing functions to sense the occurrence of position changes of people or devices in the operating room, thus triggering the active movement in response. Therefore, a special hardware forming system is required. At the same time, many factors need to be comprehensively considered when the robot is actively adjusted to the objective pose, including but not limited to: measurement accuracy, measurable conditions of target positioning, accessibility of the robot, etc. No optical positioning tool can be lost when adjusting the pose during the surgery, so that special robot pose optimization and path planning control algorithms are needed.

### SUMMARY

In view of the above factors, the present disclosure provides an active navigation system of a surgery and a control method thereof. The technical scheme of the present disclosure solves the problems of acquiring an optimal observation pose of a robot for surgical navigation and positioning, performing real-time active adjustment of the position, preventing the optical tracking system from being interfered, and improving the positioning accuracy of the navigation process, etc.

A control method of an active navigation system of the surgery described above is provided, comprising the following steps:
Step 1, measurement viewing angle multi-objective optimization: inputting position parameters of positioning tools and setting other related parameters, and solving a set of optimal measurement viewing angles through multi-objective optimization;
Step 2, multi-objective decision of a pose of a robot: according to the set of optimal measurement viewing angles, recommending, to a user, an optimal posture scheme of the robot in each link of the surgery by using a multi-objective decision algorithm; or selecting, according to the preference of the user, the optimal posture scheme of the robot in each link of the surgery;
Step 3, planning and execution of a path of the robot: according to the selected optimal pose scheme of the robot in each link of the surgery, planning the path of the robot from the current pose to the optimal pose scheme.
   Preferably, Step 1 comprises the following steps:
Step 1.1, obtaining information on and positions of all positioning tools of each link in a surgery process, and establishing a multi-objective minimization problem based on a decision variable;

x=[q₁,q₂,q₃, . . . ,q_{N}] (Formula 1)
where q₁, q₂, q₃, . . . , q_{N} are joint variables; N is the number of the joint variables; the decision variable x denotes a vector consisted of N joint variables of a robot, and the value range is the joint value range Q achievable by each joint of the robot, that is, x∈Q;
Step 1.2, defining at least two objective functions f₁ and f₂ of minimization optimization as follows:

f₁=maxₘ∥\vec{NMₘ}∥ (Formula 2)

f₂=min_{j,k∈s}−Oₘᵢₙ(j,k) (Formula 3)
where ∥\vec{NMₘ}∥ denotes the distance between the coordinate origin of the m-th positioning tool and the coordinate origin of a positioning sensor; f₁ denotes the maximum distance between the coordinate origin of all positioning tools and the coordinate origin of the positioning sensor; Oₘᵢₙ(j, k) denotes the smaller non-interference margin function in the camera coordinates of the positioning sensor for a given pair of positioning tools j and k; Min_{j,k∈s}Oₘᵢₙ(j, k) denotes the minimum non-interference margin function value among the binary combinations of all the positioning tools measured in all the cameras of the positioning sensor under the posture of the robot determined by the decision variable x;
calculating the smaller non-interference margin function Oₘᵢₙ(j, k) by the following formula:

$$\begin{matrix}{α}_{G , j , k} = {cos}^{- 1} ( \frac{\vec{{GM}_{j}} \cdot \vec{{GM}_{k}}}{‖ \vec{{GM}_{j}} ‖ ‖ \vec{{GM}_{k}} ‖} ) & ( Formula 4 ) \\ {β}_{G , j} = {sin}^{- 1} ( \frac{{r}_{j}}{‖ \vec{{GM}_{j}} ‖} ) & ( Formula 5 ) \\ {r}_{j} = ω {l}_{j} and ω > 1 & ( Formula 6 ) \\ {β}_{G , k} = {sin}^{- 1} ( \frac{{r}_{k}}{‖ \vec{{GM}_{k}} ‖} ) & ( Formula 7 ) \\ {r}_{k} = ω {l}_{k} and ω > 1 & ( Formula 8 ) \\ O ( j , k , G ) = {min}_{G = R , L} ( {α}_{G , j , k} - {β}_{G , j} - {β}_{G , k} ) & ( Formula 9 ) \\ O ( j , k , L ) = {min}_{L} ( {α}_{L , j , k} - {β}_{L , j} - {β}_{L , k} ) & ( Formula 10 ) \\ O ( j , k , R ) = {min}_{R} ( {α}_{R , j , k} - {β}_{R , j} - {β}_{R , k} ) & ( Formula 11 ) \\ {O}_{min} ( j , k ) = min ( O ( j , k , L ) , O ( j , k , R ) ) & ( Formula 12 )\end{matrix}$$

where G is the coordinate origin of the left or right camera in the positioning sensor; L and R are the coordinate origins of the left and right cameras in the positioning sensor, respectively; Mⱼ and Mₖ are the centers of the minimum circumscribed ball of any two positioning tools j and k in which the radii are lⱼ and lₖ, respectively, that is, the coordinate origin of the positioning tools j and k; rⱼ and rₖ are the extension radii of the positioning tools j and k, respectively; the margin coefficient ω is a constant greater than 1; the vector lengths ∥\vec{GM_{J}}∥ and ∥\vec{GMₖ}∥ are measured by the positioning sensor; denotes vector point multiplication;
Step 1.3, setting the following constraint conditions to minimize at least two objective functions f₁ and f₂ at the same time while ensuring that the following constraint conditions are met:

$$constraint condition 1 : ∀ i ∈ S , {M}_{i} ∈ A ( x ) \\ constraint condition 2 : ∀ i ∈ S , {max}_{G ∈ \{ R , L \}} {α}_{G , i} \leq Th \\ constraint condition 3 : ∀ i ∈ S , {min}_{G ∈ \{ R , L \}} O ( j , k , G ) \geq 0 ,$$

wherein
constraint condition 1 indicates that any positioning tool should be in the observation range of both the positioning sensor and an environmental perception sensor;
constraint condition 2 indicates that the included angle between the connecting line from the camera on either side of the positioning sensor to any positioning tool and the z-axis direction of the positioning tool is not greater than the established threshold; α_{G,i} denotes the included angle between the vector from the coordinate origin of the i-th positioning tool to the coordinate origin of the left or right camera in the positioning sensor and the vector in the z-axis direction of the i-th positioning tool; and Th is a preset threshold;
constraint condition 3 indicates that any two positioning tools are not interfered from each other, that is, the minimum value of the non-interference margin function O(j, k, G) between any two positioning tools is non-negative.
   Preferably, in Step 2, according to the set of optimal measurement viewing angles, recommending, to a user, an optimal pose scheme of the robot in each link of the surgery by using a multi-objective decision algorithm, comprises the following steps:
Step 2.1: finding out the optimal solution on a single objective in the set of optimal measurement viewing angles, and calculating the linear equation where the two endpoints of the curve corresponding to the set of optimal measurement viewing angles are located:

Af₁+Bf₂+C=0 (Formula 13)
Step 2.2: calculating the vertical distance d from each point in the curve corresponding to the set of optimal measurement angles to the straight line, and substituting the objective value of each point into the following formula:

$$d = \frac{{Af}_{1} + {Bf}_{2} + C}{\sqrt{{A}^{2} + {B}^{2}}} \qquad ( Formula 14 )$$

Step 2.3: taking the solution of the optimal measurement viewing angle corresponding to the maximum value of the vertical distance d as the recommended value of the multi-objective decision of the joint value of the robot;
where A, B and C are obtained by solving the linear equation with the objective value of the single-objective optimal solution.
   Preferably, Step 3 comprises the following steps:
Step 3.1: in a surgical process, after entering the designated surgical link, obtaining the objective posture of the current surgical link according to the optimal pose scheme obtained by optimal solution and multi-objective decision of the pose of the robot before surgery and the optimal pose scheme of the robot during surgery;
Step 3.2: obtaining, by an environmental perception sensor, the three-dimensional information of the surrounding environment of the surgical robot, generating a point cloud image C_{B} of the surrounding environment, and obtaining the point cloud position information C_{N} of the environmental point cloud under the coordinates of the positioning sensor by the following formula:

C_{N}=T_{B}^{N}C_{B} (Formula 15)
where T_{B}^{N} is a 4*4 constant transformation matrix;
Step 3.3: randomly generating candidate path points;
Step 3.4: judging whether the path point will encounter an obstacle; if so, returning to Step 3.3; otherwise, proceeding to the next step;
Step 3.5: judging whether all positioning tools are observable in this pose; if not, returning to
Step 3.3; otherwise, proceeding to the next step;
wherein in the step of judging whether all positioning tools are observable in this pose, it is required that the positioning tools meet the above constraint conditions 1-3;
Step 3.6: adding the current candidate path points to a path directory to generate a reasonable path plan;
Step 3.7: judging whether the objective pose has been reached; if not, returning to Step 3.3; otherwise, finding out the shortest path in the current path directory as the movement path of the robot;
Step 3.8: carrying out the above path pose, so that the robot of the surgical robot reaches the objective pose.
   An active navigation system of a surgery is provided, which executes the control method of the active navigation system of the surgery described above, where the system is composed of a control host, a series robot having multi degrees of freedom, a positioning sensor and one or more positioning tools adapted to the positioning sensor, and an environment perception sensor; the overlapping measurement area of the environmental perception sensor and the positioning sensor is the measurable area of the active navigation system of the surgery;
there is one or more positioning tools; each positioning tool is provided with K positioning parts which are distributed and formed according to a certain positional relationship; the positioning part is a specific marker capable of reflecting light or emitting light, and/or a part formed by arranging a plurality of specific patterns according to a certain positional relationship; the specific marker capable of reflecting light at least comprises: balls with high reflectivity coating on the surfaces; the specific marker capable of emitting light at least comprises: an LED lamp; the specific pattern is a pattern specially coded and designed, and at least comprises a QR Code and a Gray Code;
the position and/or number of each positioning tool on each positioning tool are different to distinguish the positioning tools; the centroids of K positioning parts of the same positioning tool are all on the same plane;
the center of each positioning tool is designed with a special shape feature, and the plane focus where the feature axis and the centroid of the positioning part are located is taken as the coordinate origin; the coordinate origin is taken as the center of the sphere, a minimum circumscribed ball enveloping K positioning parts on the positioning tool is constructed for each positioning tool, the radius of the minimum circumscribed ball is lᵢ; the normal direction of the plane where the centroids of K positioning parts are located is taken as the z-axis direction; the direction towards the side where the K positioning parts are attached is the positive direction of the z axis; and a three-dimensional Cartesian coordinate system is established by taking the direction perpendicular to the z axis and pointing to the positioning part farthest from the coordinate origin as the positive direction of the x axis;
the set of all positioning tools is denoted as S, in which the center of the coordinate system of the i-th positioning tool is Mᵢ, that is, Mᵢ∈S.
   In practical application, a certain margin will be added on the basis of lᵢ, that is, the spherical surface size is set slightly larger than lᵢ during estimation, for example, lᵢ is multiplied by a margin coefficient ω greater than 1 to obtain rᵢ, so as to prevent some small errors in practical operation from leading to the failure of the method.

### Beneficial Effects:

The present disclosure provides an active navigation system of a surgery and a control method thereof. The technical scheme of the present disclosure solves the problem of acquiring an optimal observation pose of a robot for surgical navigation and positioning, performing real-time active adjustment of the position, preventing the navigation target locator from being blocked, and improving the positioning accuracy of the navigation process, etc.

### BRIEF DESCRIPTION OF THE DRAWINGS

FIG. 1 is an overall structural diagram of an active navigation system of surgery according to the present disclosure.

FIG. 2 is an embodiment diagram of an active navigation system of surgery according to the present disclosure.

FIG. 3 is a schematic diagram of establishing a coordinate system in an active navigation system of surgery according to the present disclosure.

FIG. 4 is a diagram of establishing a positioning tool and a coordinate system thereof according to the present disclosure.

FIG. 5 is a schematic diagram of the design of an non-occlusion margin function O(j, k, G) according to the present disclosure.

FIG. 6 is a schematic diagram of an observation angle α_{G,i} according to the present disclosure.

FIG. 7 is an optimal solution diagram of the measurement viewing angle multi-objective optimization according to the present disclosure.

FIG. 8 is a diagram of an optimal solution recommendation method provided by a multi-objective decision algorithm according to the present disclosure.

### DETAILED DESCRIPTION

The technical scheme in the embodiment of the present disclosure will be described clearly and completely with reference to the attached drawings hereinafter.

The present disclosure provides an active navigation system of a surgery and a control method thereof.

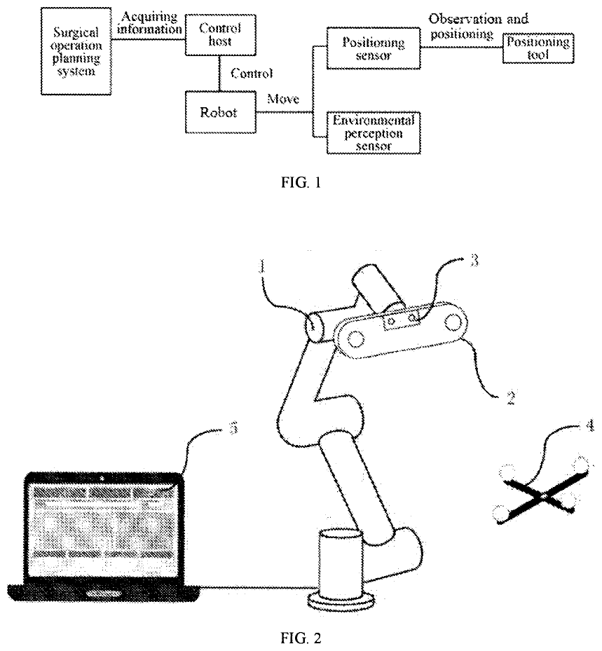

FIG. 1 is an overall structural diagram of an active navigation system of a surgery according to the present disclosure. As shown in FIG. 1, the system comprises a surgical operation planning system, a control host for data processing and robot control, a robot, a positioning sensor and adaptive positioning tools thereof, and an environmental perception sensor; the environment perception sensor realizes the sensing of the surgical environment, such as potential obstructions and/or obstacles. The robot is a serial robot with 7 degrees of freedom; the positioning sensor and/or the environment perception sensor are connected to an flange of the robot.

The positioning sensor can use many different modes, such as a binocular depth camera based on visible light, a binocular positioning camera based on near-infrared light, etc. The corresponding positioning tool is an optical QR Code or another coded pattern matched with the positioning sensor, or a positioning tool consisted of optical balls whose surfaces are covered with special paint, etc.

The environmental perception sensors can also use many modes, such as a binocular depth camera based on visible light, a laser radar, an ultrasonic sensor, etc.

The environmental perception sensor and the positioning sensor can be the combination of two device carriers, such as the scheme of a binocular positioning camera and a laser radar based on near-infrared light; the sensors can also be the same type of sensors, such as a binocular depth camera based on visible light, which can be used for positioning and realizing surgical environment perception. However, in any case, the spatial areas measured by the environmental perception sensor and the positioning sensor must be mutually overlapping areas, and the mutually overlapping areas are the measurable areas of the system.

FIG. 2 is an embodiment diagram of an active navigation system of surgery according to the present disclosure. As shown in FIG. 2, the implementation is as follows. The system consists of a robot with 7 degrees of freedom, a near-infrared optical positioning system (as a “positioning sensor”) and a binocular camera (as an environmental perception sensor) connected to the flange of the robot, a computer for data processing and robot control, and a positioning tool adapted to the near-infrared optical positioning system.

The near-infrared optical positioning system here includes two infrared emitting lamps and an infrared camera for detecting reflected infrared light. The working principle is that the left and right infrared emitting lamps emit specific infrared light and project the specific infrared light on the surface of a reflective ball on the positioning tool. The reflective ball reflects infrared light, which is detected by the infrared camera. According to the received reflected infrared light, the relative position between the near-infrared optical positioning system and each ball is calculated, and the relative position of each positioning tool with respect to the near-infrared optical positioning system is calculated according to the pre-calibrated positioning relationship model.

The base coordinate of the robot is 0, the joint angle of the kth joint is qₖ, and the origin of the coordinate system of the flange is {E}. The center coordinate of the near-infrared optical positioning system is {N}, and the coordinates of the left and right cameras are R and L, respectively. When the robot is in position p, the measurable area space of the near-infrared optical positioning system is A(p). The coordinate system of the binocular camera is {C}.

As shown in FIG. 2, the reference numerals have the following meanings: 1. Robot with 7 degrees of freedom, 2. Near-infrared optical positioning system, 3. Binocular camera, 4. Positioning tool, and 5. Computer.

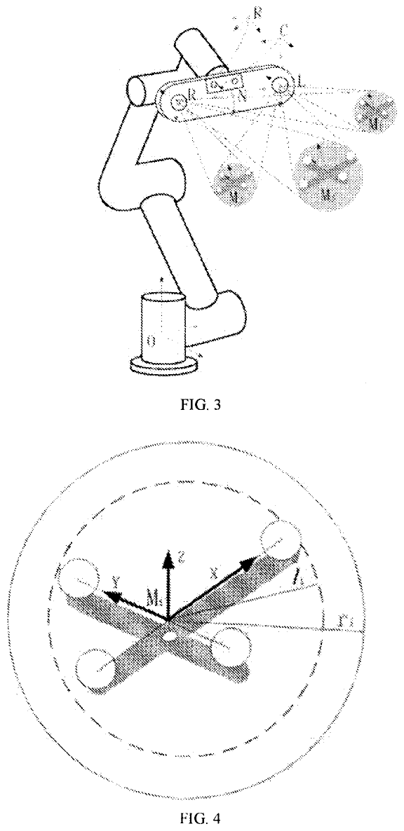

FIG. 3 is a schematic diagram of establishing a coordinate system in a surgical robot navigation and positioning system according to the present disclosure. The set of all positioning tools is S. The center of the coordinate system of the i-th positioning tool is Mᵢ, that is, Mᵢ∈S. The center coordinate of the optical positioning system is N, and the coordinates of the left and right cameras are R and L, respectively. When the robot is in position p, the measurable area space where the optical positioning system and the environmental perception sensor overlap is A(p), that is, the set of all possible positions where the positioning tool can be measured normally when the robot is in position p without occlusion. The coordinate system of the binocular camera is C.

FIG. 4 is a diagram of establishing a positioning tool and a coordinate system thereof according to the present disclosure. The positioning tool is matched with the near-infrared optical positioning system (i.e. “positioning sensor”), as shown in FIG. 4. Each positioning tool has four balls with high reflectivity coating on the surface, which are distributed according to a certain positional relationship. The centers of four balls of the same positioning tool are on the same plane, and the normal direction of the plane where the centroid of K positioning parts is located is the z-axis direction; and the direction towards the side where the K positioning parts are attached is the positive direction of the z axis. The position and/or number of balls of each positioning tool are different to distinguish the positioning tools. Each positioning tool uses the plane where the center of the ball is located. The intersection point with the central axis of the central hole of the connecting rod of the positioning tool (that is, an example of shape features) is taken as the coordinate origin, and the direction of the intersection point pointing to the ball farthest from the origin is taken as the x-axis direction. Taking the intersection point as the center of the circle, the minimum circumscribed ball enveloping all the balls is established, and the radius of the circumscribed ball is lᵢ. The set of all positioning tools is S. The center of the coordinate system of the i-th positioning tool is Mᵢ, that is, Mᵢ∈S.

The present disclosure provides a control method of an active navigation system of a surgical robot. The control method comprises three parts: “measurement viewing angle multi-objective optimization”, “multi-objective decision of a pose of robot”, and “planning and execution of a path of the robot”. The details are as follows.
measurement viewing angle multi-objective optimization: the state and position of the positioning tools are input into the program, the relevant parameters are set, and then a set of optimal measurement viewing angles is solved through multi-objective optimization.
multi-objective decision of a posture of a robot: based on the optimal solution set obtained by optimization in the previous step, a multi-objective decision algorithm is used to recommend the scheme to the user, or the user selects the appropriate pose scheme of the robot for surgical navigation according to the preference in each link of the surgery.
planning and execution of a path of the robot: based on the optimal pose scheme in each link of the surgery obtained in the previous step, the robot plans the scheme from the current pose to the optimal pose through an algorithm. In this process, it is necessary to consider that the positioning sensor can always position all the positioning tools needed in the surgical link normally during the movement, and consider unexpected obstacles in this process. Finally, the appropriate optimal pose is achieved.

The contents of the above three parts are introduced as follows.
(1) Measurement viewing angle multi-objective optimization: information on and positions of all positioning tools of each link in a surgery process are obtained through the surgical operation planning system. The following multi-objective minimization problem is established: a decision variable: x=[q₁, q₂, q₃, . . . , q_{N}]
where q₁, q₂, q₃, . . . , q_{N} are joint variables; N is the number of the joint variables; the decision variable x denotes a vector consisted of N joint variables of a robot, and the value range is the joint value range Q achievable by each joint of the robot, that is, x∈Q;
   The optimization objective is as follows (at least two objective functions f₁ and f₂ are simultaneously minimized).
   Optimization objective 1: the maximum distance between the positioning tool and the near-infrared optical positioning system is minimized;

f₁=maxₘ∥\vec{NMₘ}∥

where ∥\vec{NMₘ}∥ denotes the distance between the coordinate origin of the m-th positioning tool and the coordinate origin of the near-infrared optical positioning system.
Optimization objective 2: min_{j,k∈S}Oₘᵢₙ(j, k) denotes the minimum non-interference margin function value between the positioning tools. By taking the inverse number of its value, the value is transformed into a minimization optimization problem:

f₂=min_{j,k∈S}−Oₘᵢₙ(j,k)
where Oₘᵢₙ(j, k) denotes the smaller non-interference margin function in the camera coordinates of the positioning sensor for a given pair of positioning tools j and k; min_{j,k∈S}Oₘᵢₙ(j, k) denotes the minimum non-interference margin function value among the binary combinations of all the positioning tools measured in all the cameras of the positioning sensor under the pose of the robot determined by q;
the non-interference margin function O(j, k, G) between the positioning tools j and k is defined as shown in FIG. 5.
   FIG. 5 is a schematic diagram of the design of an non-interference margin function O(j, k, G) according to the present disclosure, which describes the definition of the non-interference margin function O(j, k, G). Specifically, FIG. 5 describes the geometric relationship between any two positioning tools and either of the left camera or the right camera of the positioning sensor. Therefore, if the number of the positioning tools is greater than 2, any two positioning tools and either of the left camera or the right camera will generate a specific value of O(j, k, G). For example, three positioning tools can generate six O(j, k, G) values, namely: O(1, 2, L), O(1, 3, L), O(2, 3, L), O(1, 2, R), O(1, 3, R), O(2, 3, R).
G is the coordinate origin of the left or right camera in the positioning sensor. Mⱼ and Mₖ are the centers of any two positioning tools after the two positioning tools are abstracted into spheres, and are also the origin of the coordinate system of the positioning tools. rⱼ and rₖ are the radii of the sphere into which the positioning tools are abstracted. Each positioning tool uses the intersection point between the plane where the center of the ball is located and the central axis of the central hole (that is, an example of shape features) of the connecting rod of the positioning tool as the coordinate origin. The minimum circumscribed ball radius with the coordinate origin as the center is Considering the influence of errors in actual operation, the radius rᵢ and rₖ of the sphere into which the positioning tools are abstracted is obtained by expanding the margin co times on the basis of (The feature of the positioning tool here is that four or more coplanar connecting rods extend from a center, and the ends of the connecting rods are provided with balls. In a set of navigation devices, the relative position between the balls of each positioning tool is unique). ω>1.
   Therefore, the size of rⱼ and rₖ are known. The length of the vectors ∥\vec{GM_{J}}∥ and ∥\vec{GMₖ}∥ can be measured by the positioning sensors. βG,j and βG,k can be obtained by the following relationship:

$${β}_{G , j} = {sin}^{- 1} ( \frac{{r}_{j}}{‖ \vec{{GM}_{j}}} ) \\ {β}_{G , k} = {sin}^{- 1} ( \frac{{r}_{k}}{‖ \vec{{GM}_{k}} ‖} )$$

α_{G,j,k} can be calculated by the vector:

$${α}_{G , j , k} = {cos}^{- 1} ( \frac{\vec{{GM}_{j}} \cdot \vec{{GM}_{k}}}{‖ \vec{{GM}_{j}} ‖ ‖ \vec{{GM}_{k}} ‖} )$$

where denotes the vector point multiplication.

### Finally,

$$O ( j , k , G ) = {min}_{G = R , L} ( {α}_{G , j , k} - {β}_{G , j} - {β}_{G , k} )$$

is calculated,
where rᵢ=ωlᵢ, which denotes the radius of the sphere after the positioning tool is abstracted and simplified; where ω>1.
   The constraint conditions are as follows:

$$constraint condition 1 : ∀ i ∈ S , {M}_{i} ∈ A ( x ) \\ constraint condition 2 : ∀ i ∈ S , {max}_{G ∈ \{ R , L \}} {α}_{G , i} \leq Th \\ constraint condition 3 : ∀ i ∈ S , {min}_{G ∈ \{ R , L \}} O ( j , k , G ) \geq 0 ,$$

wherein
constraint condition 1 indicates that any positioning tool should be in the observable range of both the positioning sensor and the environmental perception sensor;
constraint condition 2 indicates that the included angle between the connecting line from the camera on either side of the positioning sensor to any positioning tool and the z-axis direction of the positioning tool is not greater than the established threshold; α_{G,i} denotes the included angle between the vector from the coordinate origin of the i-th positioning tool to the coordinate origin of the left or right camera in the positioning sensor and the vector in the z-axis direction of the i-th positioning tool; and Th is a preset threshold, for example, Th=π/2;
constraint condition 3 indicates that any two positioning tools are not interfered from each other, that is, the minimum value of the non-interference margin function O(j, k, G) between any two positioning tools is non-negative.

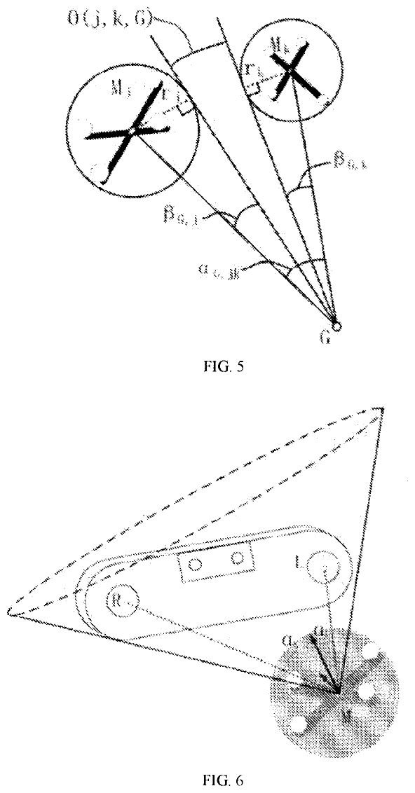

FIG. 6 is a schematic diagram of an observation angle α_{G,i} according to the present disclosure. The observation angle refers to the included angle between the origin of the left or right camera and the z axis of any of the positioning tools (the normal direction of the positioning tool pointing upward is fixed as the z axis of the coordinate of the positioning tool).

$${α}_{G , i} = {cos}^{- 1} ( \frac{\vec{{GM}_{1}} \cdot \vec{Z}}{‖ \vec{{GM}_{1}} ‖ ‖ \vec{Z} ‖} )$$

As shown in FIG. 6, {G} is the origin of the coordinate system of the left or right camera of the positioning sensor. \vec{Z} is the Z-axis unit vector of the positioning tool in {G}. \vec{Z} and \vec{GM₁} can be obtained by the positioning sensor so as to be substituted into the formula for calculation. In addition, it should be noted that any camera on either side will have an observation angle value for any positioning tool.

To sum up, the following optimization problems need to be optimized:
The decision variable: x=[q₁, q₂, q₃, . . . , q_{N}]
At the same time:

f₁=maxₘ∥\vec{NMₘ}∥

f₂=min_{j,k∈S}−Oₘᵢₙ(j,k)

is minimized.
At the same time, the following constraint conditions are considered:

$$\begin{matrix}∀ i ∈ S , {M}_{i} ∈ A ( x ) & ( i ) \\ ∀ i ∈ S , {max}_{G ∈ \{ R , L \}} {α}_{G , i} \leq Th & ( ii ) \\ ∀ i ∈ S , {min}_{G ∈ \{ R , L \}} O ( j , k , G ) \geq 0 & ( iii )\end{matrix}$$

The above optimization problems can be solved by constraining the multi-objective optimization algorithm. In this embodiment, the Pareto optimal solution of the above optimization problem can be obtained by using the MOEA/D-CDP algorithm.

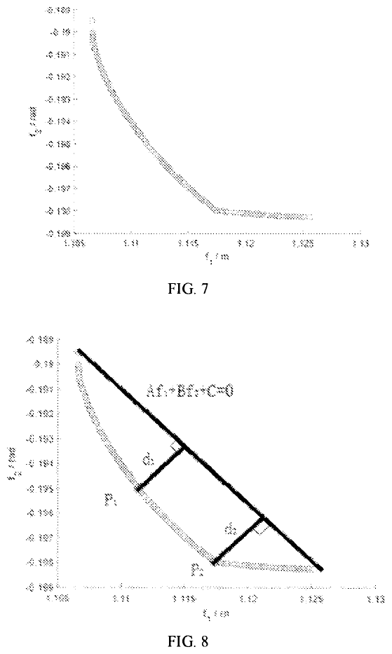

FIG. 7 is an optimal solution diagram of the measurement viewing angle multi-objective optimization according to the present disclosure.

As shown in FIG. 7, each point in the figure corresponds to an optimized pose scheme. These schemes do not dominate each other, and they are all optimal solutions.

### (2) Multi-Objective Decision of a Posture of a Robot.

After obtaining the optimal solution of measurement viewing angle multi-objective optimization as shown in FIG. 7, users can directly select any of the above optimal solutions according to their own preferences; or select after making a recommendation based on the multi-objective decision algorithm provided by the system.
FIG. 8 is a diagram of an optimal solution recommendation method provided by a multi-objective decision algorithm according to the present disclosure.
The specific steps of the optimal solution recommendation method are as follows:
Step 1: finding out the optimal solution on a single objective in the optimal solution set, and calculating the linear equation where the two endpoints are located:

Af₁+Bf₂+C=0
Step 2: calculating the vertical distance d from each point to the straight line, and substituting the objective value of each point into the following formula:

$$d = \frac{{Af}_{1} + {Bf}_{2} + C}{\sqrt{{A}^{2} + {B}^{2}}}$$

Step 3: recommending the optimal solution with the maximum d value as the recommended value according to the needs of the user, so as to be used directly; or recommending several optimal solutions, so as to be selected by the user.

### (3) Planning and Execution of a Path of the Robot.

Step 1: In the specific surgical process, after entering the designated surgical link, the objective pose of the current surgical link is obtained according to the optimal pose scheme obtained by optimal solution and multi-objective decision of the pose of the robot before surgery and the optimal pose scheme of the robot during surgery (that is, the optimal objective selected in the multi-objective decision link of the posture of the robot).
Step 2: The binocular camera obtains the three-dimensional information of the surrounding environment of the robot, generates a point cloud image C_{B} of the surrounding environment, and uses the following formula:

C_{N}=T_{B}^{N}C_{B}

to obtain the point cloud position information C_{N} of the environmental point cloud under the coordinates of the optical positioning system; where T_{B}^{N} is a 4*4 constant transformation matrix, the value of which is related to the relative position of the binocular camera and the optical positioning system.
Step 3: The algorithm randomly generates candidate path points.
Step 4: It is judged whether the path point will encounter an obstacle; if so, return to Step 3.3; otherwise, proceed to the next step.
Step 5: It is judged whether all positioning tools are observable in this pose; if not, return to Step 3.3; otherwise, proceed to the next step.
   In the step of judging whether all positioning tools are observable, it is required that the positioning tools meet the above constraint conditions:

$$\begin{matrix}∀ i ∈ S , {M}_{i} ∈ A ( x ) & ( i ) \\ ∀ i ∈ S , {max}_{G ∈ \{ R , L \}} {α}_{G , i} \leq Th & ( ii ) \\ ∀ i ∈ S , {min}_{G ∈ \{ R , L \}} O ( j , k , G ) \geq 0 & ( iii )\end{matrix}$$

Step 6: The current candidate path points are added to a path directory to finally generate a reasonable path plan.
Step 7: It is judged whether the objective pose has been reached; if not, return to Step 3.3; otherwise, the shortest path in the current path directory is found out as the movement path of the robot.
Step 8: The above path pose is carried out, so that the robot reaches the objective pose.
   The above is only a specific embodiment of the present disclosure, but the scope of protection of the present disclosure is not limited thereto. Various equivalent modifications or substitutions conceivable to those skilled in the art within the technical scope disclosed by the present disclosure should be included in the scope of protection of the present disclosure. Therefore, the scope of protection of the present disclosure shall be subject to the scope of protection of the claims.

1. A control method of an active navigation system of surgery, comprising the following steps:
Step 1, measurement viewing angle multi-objective optimization: inputting position parameters of positioning tools and setting other related parameters, and solving a set of optimal measurement viewing angles through multi-objective optimization;
Step 2, multi-objective decision of a pose of a robot: according to the set of optimal measurement viewing angles, recommending, to a user, an optimal pose scheme of the robot in each link of the surgery by using a multi-objective decision algorithm; or selecting, according to the preference of the user, the optimal pose scheme of the robot in each link of the surgery;
Step 3, planning and execution of a path of the robot: according to the selected optimal pose scheme of the robot in each link of the surgery, planning the path of the robot from the current pose to the optimal pose scheme;
wherein Step 1 comprises the following steps:
Step 1.1, obtaining information on and positions of all positioning tools of each link in a surgery process, and establishing a multi-objective minimization problem based on a decision variable;
x=[q₁,q₂,q₃, . . . ,q_{N}] (Formula 1)
where q₁, q₂, q₃, . . . , q_{N} are joint variables; N is the number of the joint variables; the decision variable x denotes a vector consisted of N joint variables of a robot, and the value range is the joint value range Q achievable by each joint of the robot, that is, x∈Q;
Step 1.2, defining at least two objective functions f₁ and f₂ of minimization optimization as follows:
f₁=maxₘ∥\vec{NMₘ}∥ (Formula 2)
f₂=min_{j,k∈S}−Oₘᵢₙ(j,k) (Formula3)
where ∥\vec{NMₘ}∥ denotes the distance between the coordinate origin of the m-th positioning tool and the coordinate origin of a positioning sensor; f₁ denotes the maximum distance between the coordinate origin of all positioning tools and the coordinate origin of the positioning sensor; Oₘᵢₙ(j, k) denotes the smaller non-interference margin function in the camera coordinates of the positioning sensor for a given pair of positioning tools j and k; min_{j,k∈S}Oₘᵢₙ(j, k) denotes the minimum non-interference margin function value among the binary combinations of all the positioning tools measured in all the cameras of the positioning sensor under the posture of the robot determined by the decision variable x;
calculating the smaller non-interference margin function Oₘᵢₙ(j, k) by the following formula:
$$\begin{matrix}{α}_{G , j , k} = {cos}^{- 1} ( \frac{\vec{{GM}_{j}} \cdot \vec{{GM}_{k}}}{‖ \vec{{GM}_{j}} ‖ ‖ \vec{{GM}_{k}} ‖} ) & ( Formula 4 ) \\ {β}_{G , j} = {sin}^{- 1} ( \frac{{r}_{j}}{‖ \vec{{GM}_{j}} ‖} ) & ( Formula 5 ) \\ {r}_{j} = ω {l}_{j} and ω > 1 & ( Formula 6 ) \\ {β}_{G , k} = {sin}^{- 1} ( \frac{{r}_{k}}{‖ \vec{{GM}_{k}} ‖} ) & ( Formula 7 ) \\ {r}_{k} = ω {l}_{k} and ω > 1 & ( Formula 8 ) \\ O ( j , k , G ) = {min}_{G = R , L} ( {α}_{G , j , k} - {β}_{G , j} - {β}_{G , k} ) & ( Formula 9 ) \\ O ( j , k , L ) = {min}_{L} ( {α}_{L , j , k} - {β}_{L , j} - {β}_{L , k} ) & ( Formula 10 ) \\ O ( j , k , R ) = {min}_{R} ( {α}_{R , j , k} - {β}_{R , j} - {β}_{R , k} ) & ( Formula 11 ) \\ {O}_{min} ( j , k ) = min ( O ( j , k , L ) , O ( j , k , R ) ) & ( Formula 12 )\end{matrix}$$
where G is the coordinate origin of the left or right camera in the positioning sensor; L and R are the coordinate origins of the left and right cameras in the positioning sensor, respectively; lⱼ and lₖ are the radii of the minimum circumscribed ball of any two positioning tools j and k, Mⱼ and Mₖ are the centers of the minimum circumscribed ball of any two positioning tools j and k, respectively, that is, the coordinate origin of the positioning tools j and k; rⱼ and rₖ are the extension radii of the positioning tools j and k, respectively; the margin coefficient ω is a constant greater than 1; the vector lengths ∥\vec{GMⱼ}∥ and ∥\vec{GMₖ}∥ are measured by the positioning sensor; · denotes vector point multiplication;
Step 1.3, setting the following constraint conditions to minimize at least two objective functions f₁ and f₂ at the same time while ensuring that the following constraint conditions are met:
$$constraint condition 1 : ∀ i ∈ S , {M}_{i} ∈ A ( x ) \\ constraint condition 2 : ∀ i ∈ S , {max}_{G ∈ \{ R , L \}} {α}_{G , i} \leq Th \\ constraint condition 3 : ∀ i ∈ S , {min}_{G ∈ \{ R , L \}} O ( j , k , G ) \geq 0 ,$$
wherein
constraint condition 1 indicates that any positioning tool should be in the observable range of both the positioning sensor and an environmental perception sensor;
constraint condition 2 indicates that the included angle between the connecting line from the camera on either side of the positioning sensor to any positioning tool and the z-axis direction of the positioning tool is not greater than the established threshold; α_{G,i} denotes the included angle between the vector from the coordinate origin of the i-th positioning tool to the coordinate origin of the left or right camera in the positioning sensor and the vector in the z-axis direction of the i-th positioning tool; and Th is a preset threshold;
constraint condition 3 indicates that any two positioning tools are not interfered from each other, that is, the minimum value of the non-interference margin function O(j, k, G) between any two positioning tools is non-negative.

2. The control method according to claim 1, wherein in Step 2, according to the set of optimal measurement viewing angles, recommending, to a user, an optimal posture scheme of the robot in each link of the surgery by using a multi-objective decision algorithm, comprises the following steps:
Step 2.1: finding out the optimal solution on a single objective in the set of optimal measurement viewing angles, and calculating the linear equation where the two endpoints of the curve corresponding to the set of optimal measurement viewing angles are located:
Af₁+Bf₂+C=0 (Formula 13)
Step 2.2: calculating the vertical distance d from each point in the curve corresponding to the set of optimal measurement angles to the straight line, and substituting the objective value of each point into the following formula:
$$d = \frac{{Af}_{1} + {Bf}_{2} + C}{\sqrt{{A}^{2} + {B}^{2}}} \qquad ( Formula 14 )$$
Step 2.3: taking the solution of the optimal measurement viewing angle corresponding to the maximum value of the vertical distance d as the recommended value of the multi-objective decision of the joint value of the robot;
where A, B and C are obtained by solving the linear equation with the objective value of the single-objective optimal solution.

3. The control method according to claim 2, wherein Step 3 comprises the following steps:
Step 3.1: in a surgical process, after entering the designated surgical link, obtaining the objective pose of the current surgical link according to the optimal pose scheme obtained by optimal solution and multi-objective decision of the pose of the robot before surgery and the optimal pose scheme of the robot during surgery;
Step 3.2: obtaining, by an environmental perception sensor, the three-dimensional information of the surrounding environment of the surgical robot, generating a point cloud image C_{B} of the surrounding environment, and obtaining the point cloud position information C_{N} of the environmental point cloud under the coordinates of the positioning sensor by the following formula:
C_{N}=T_{B}^{N}C_{B} (Formula 15)
where T_{B}^{N} is a 4*4 constant transformation matrix;
Step 3.3: randomly generating candidate path points;
Step 3.4: judging whether the path point will encounter an obstacle; if so, returning to Step 3.3; otherwise, proceeding to the next step;
Step 3.5: judging whether all positioning tools are observable in this pose; if not, returning to Step 3.3; otherwise, proceeding to the next step;
wherein in the step of judging whether all positioning tools are observable in this pose, it is required that the positioning tools meet the above constraint conditions 1-3;
Step 3.6: adding the current candidate path points to a path directory to generate a reasonable path plan;
Step 3.7: judging whether the objective pose has been reached; if not, returning to Step 3.3; otherwise, finding out the shortest path in the current path directory as the movement path of the robot;
Step 3.8: carrying out the above path pose, so that the robot of the surgical robot reaches the objective pose.

4. An active navigation system of a surgery, which executes the control method of the active navigation system of the surgery according to any of claim 1, wherein the system comprises: a control host, a series robot having multi degrees-of-freedom, a positioning sensor and one or more positioning tools adapted to the positioning sensor, and an environment perception sensor; the overlapping measurement area of the environmental perception sensor and the positioning sensor is the measurable area of the active navigation system of the surgery;
there is one or more positioning tools; each positioning tool is provided with K positioning parts which are distributed and formed according to a certain positional relationship; the positioning part is a specific marker capable of reflecting light or emitting light, and/or a part formed by arranging a plurality of specific patterns according to a certain positional relationship; the specific marker capable of reflecting light at least comprises: balls with high reflectivity coating on the surfaces; the specific marker capable of emitting light at least comprises: an LED lamp; the specific pattern is a pattern specially coded and designed, and at least comprises a QR Code and a Gray Code;
the position and/or number of each positioning tool on each positioning tool are different to distinguish the positioning tools; the centroids of K positioning parts of the same positioning tool are all on the same plane;
the center of each positioning tool is designed with a special shape feature, and the plane focus where the feature axis and the centroid of the positioning part are located is taken as the coordinate origin; the coordinate origin is taken as the center of the sphere, a minimum circumscribed ball enveloping K positioning parts on the positioning tool is constructed for each positioning tool, the radius of the minimum circumscribed ball is lᵢ; the normal direction of the plane where the centroids of K positioning parts are located is taken as the z-axis direction; the direction towards the side where the K positioning parts are attached is the positive direction of the z axis; and a three-dimensional Cartesian coordinate system is established by taking the direction perpendicular to the z axis and pointing to the positioning part farthest from the coordinate origin as the positive direction of the x axis;
the set of all positioning tools is denoted as S, in which the center of the coordinate system of the i-th positioning tool is Mᵢ, that is, Mᵢ∈S.

5. The active navigation system according to claim 4, wherein the shape feature is a round hole, a hemisphere, a boss or a cone.

6. An active navigation system of a surgery, which executes the control method of the active navigation system of the surgery according to any of claim 3, wherein the system comprises: a control host, a series robot having multi degrees-of-freedom, a positioning sensor and one or more positioning tools adapted to the positioning sensor, and an environment perception sensor; the overlapping measurement area of the environmental perception sensor and the positioning sensor is the measurable area of the active navigation system of the surgery;
there is one or more positioning tools; each positioning tool is provided with K positioning parts which are distributed and formed according to a certain positional relationship; the positioning part is a specific marker capable of reflecting light or emitting light, and/or a part formed by arranging a plurality of specific patterns according to a certain positional relationship; the specific marker capable of reflecting light at least comprises: balls with high reflectivity coating on the surfaces; the specific marker capable of emitting light at least comprises: an LED lamp; the specific pattern is a pattern specially coded and designed, and at least comprises a QR Code and a Gray Code;
the position and/or number of each positioning tool on each positioning tool are different to distinguish the positioning tools; the centroids of K positioning parts of the same positioning tool are all on the same plane;
the center of each positioning tool is designed with a special shape feature, and the plane focus where the feature axis and the centroid of the positioning part are located is taken as the coordinate origin; the coordinate origin is taken as the center of the sphere, a minimum circumscribed ball enveloping K positioning parts on the positioning tool is constructed for each positioning tool, the radius of the minimum circumscribed ball is lᵢ; the normal direction of the plane where the centroids of K positioning parts are located is taken as the z-axis direction; the direction towards the side where the K positioning parts are attached is the positive direction of the z axis; and a three-dimensional Cartesian coordinate system is established by taking the direction perpendicular to the z axis and pointing to the positioning part farthest from the coordinate origin as the positive direction of the x axis;
the set of all positioning tools is denoted as S, in which the center of the coordinate system of the i-th positioning tool is Mᵢ, that is, Mᵢ∈S.

7. The active navigation system according to claim 6, wherein the shape feature is a round hole, a hemisphere, a boss or a cone.
